(19) United States
(12) Patent Application Publication
(10) Pub. No.: US 2024/0188973 A1
(43) Pub. Date: Jun. 13, 2024

(54) NOISE DAMPENING DEVICE FOR MEDICAL AND NON-MEDICAL ACOUSTIC WAVES APPLICATIONS

(71) Applicant: Sanuwave, Inc., Eden Prairie, MN (US)

(72) Inventors: Iulian Cioanta, Milton, GA (US); Cary McGhin, Sugar Hill, GA (US); John Jackson, Buford, GA (US)

(73) Assignee: Sanuwave, Inc., Eden Prairie, MN (US)

(21) Appl. No.: 18/063,140

(22) Filed: Dec. 8, 2022

(51) Int. Cl. A61B 17/22; G10K 11/00

(52) U.S. Cl. CPC A61B 17/22004; G10K 11/002

(57) ABSTRACT

An acoustic wave applicator for medical and non-medical application of acoustic waves, including shockwave applications, couples to a sound dampening device that reduces noise produced by an acoustic wave source that is sealed with fluid in the applicator by an applicator membrane.

### BACKGROUND OF THE INVENTION

Any type of acoustic waves (shockwaves, pressure waves, radial waves, pseudo-planar waves, focused or non-focused waves, ultrasound, etc.) produce a certain type of noise at their generation and usage, noise that propagates in the surrounding environment. Depending on the input energy and the construction of the acoustic wave delivery applicators, the noise can have a lower or a higher decibel value. Regardless, a reduction in noise can be welcomed by the operators or the beneficiaries of the acoustic wave technologies. Both the noise and the acoustic waves travel with 300 m/s in air, 1500 m/s in fluids and more than 9000 m/s in solids, and thus the reduction in noise that travel together with the acoustic waves can be accomplished by partially collecting the noise and then redirected it backwards to the noise source. By doing that a large percentage of the initial noise is extracted and prevented to go into surrounding environment and also the collected noise that is directed backwards towards the source can cancel the subsequent noise produced at the same source.

When acoustic waves are used for medical applications, in urology, orthopedics, cardiovascular, wound care, inflammations, bacterial or fungal infections, etc., to name a few, the actual medical treatments are usually done indoors and in rooms with limited space, which can amplify the noise of the acoustic waves that are produced and employed for the medical procedure. That can affect negatively both the medical personnel and the patients. Thus, reducing the noise is necessary, to assure a better comfort for medical staff and patients. Also, for the medical personnel a noise reduction will help to prevent any work hazards as hearing impairment, since they use such technology repeatedly.

For the non-medical applications, where acoustic waves can be used for production processes, disinfection, ship hull cleaning, underwater structures cleaning, microorganisms and biofilms removal, meat processing, water cleaning, pipes unclogging, desalinization, filter cleaning, etc., to name a few, a reduction in noise is always welcomed. The noise can be critical for the marine life, wild animals, operators, environmental background noise, etc. Also, when compared to the medical applications, for non-medical industrial processes that use the acoustic waves technology, in general larger and more powerful acoustic waves producing systems are used that produce more noise. Furthermore, to be efficient industrial settings use multiple acoustic wave system in parallel, which is further amplifying the noise produced during functioning. Consequently, the amount of noise produced by industrial settings can be significant higher and even more annoying to the operators when compared to the medical systems. Thus, reducing the noise is also necessary in non-medical applications, to protect the environment and the living beings that populate it, humans and animals.

In general, the acoustic waves are produced in a specific element or system or device known as applicator or wand or probe or emitter, etc. that allows the generation of the acoustic waves using a certain principle/method and their unfocused or focused transmission towards the targeted zone. From this point on, applicator will be used interchangeable to define such element or system or device that produces acoustic waves (shockwaves, pressure waves, radial waves, pseudo-planar waves, focused or non-focused waves, ultrasound, etc.).

To reduce the noise produced by acoustic waves (shockwaves, pressure waves, radial waves, pseudo-planar waves, focused or non-focused waves, ultrasound, etc.), the generic or specially designed applicators should be capable to receive and accommodate snuggly a sound or noise dampening device or element or component or part, which will be presented in detail in the embodiments of these present inventions from this patent. The sound or noise dampening device being independent from the applicators and not integral part of them, assures interchangeability and portability from one installation to another and in case of malfunction reduces the cost of replacement. Finally, such noise dampening devices are designed with simplicity in mind and do not have any moving parts, which increases their reliability and assures an easy maintenance.

### SUMMARY OF THE INVENTION

Shockwaves are audible and strong pressure impulses in any elastic medium (air, water, or solids) created by supersonic crafts, lightning, explosions, earthquakes, or other extreme phenomena that generate sudden and significant changes in pressure. Shockwaves can be produced in a fluid and tailored to not produce damage to the targeted surfaces. To provide different energy transfer mechanisms for generating focused acoustic pressure shockwaves, the following means can be used:
electrohydraulic generators using high voltage discharges
electrohydraulic generators using one or multiple laser sources
piezoelectric generators using piezoelectric crystals
piezoelectric generators using piezoelectric fibers
electromagnetic generators using a flat coil
electromagnetic generators using a cylindrical coil
explosive generators that use explosive charges
hydraulic generators that use pressurized fluids exiting through special predefined orifices

The shockwaves can be oriented and pushed uni-directional via a “reflector”, which geometrically can be half of an ellipsoid or a paraboloid, or a sphere, or a pyramid, or a cone, or flat surface, etc. or any combination of these different geometries. The geometry of the reflector will dictate the focusing or non-focusing of the shockwaves towards the region of interest. For the ellipsoidal reflector and electrohydraulic generators, the shockwaves are produced in its first focal point and then focused towards second focal point of the ellipsoid, which is often described as the “focal point”. In reality due to the delay in between different direct or reflected shockwave fronts, the focusing is done in a “focal volume” and not only in a focal point. The focal volume is the region that encompasses the highest pressures generated by shockwaves and where the main activity for the shockwaves will happen. If paraboloid reflectors are used in combination with electromagnetic or piezoelectric generators, focused shockwaves can also be produced in the targeted area, if the focal point of the paraboloid geometry is outside the paraboloidal reflector. If the focal point of the paraboloid geometry is inside the reflector, then unfocused shockwave or pressure waves are produced. The unfocused shockwaves or any kind of pressure waves (planar, pseudo-planar, radial, etc.) in general produce less powerful acoustic waves with less energy carried in them, when compared to the focused shockwaves.

If the reflector is a paraboloid and the generator is using electrohydraulic principle and the pressure waves are produced in the focal point of the paraboloid that is inside the reflector, then the acoustic waves are reflected from the surface of the reflector in a parallel manner relative to the longitudinal axis of the reflector, creating pseudo-planar pressure waves (not shockwaves per se) that are unfocused. If the reflector is a sphere and an electrohydraulic generator is used, then the pressure waves can be created in the center of the sphere and then pushed out of the reflector as radial pressure waves, which are also unfocused.

Sometimes combination geometries can be used for the reflectors mentioned in the present inventions. Two or more geometries can be used as portion of an ellipsoid, combined with a portion of a sphere and a portion of a paraboloid, to give one example. That can have an effect on the way the shockwaves or pressure waves or ultrasound are reflected, how many focal volumes or pressure fields are created that can overlap or can be totally separated, and finally the actual focal volume or pressure field shape and its position in space.

Due to uni-directional propagation of the acoustic shockwaves and pressure waves, also the noise associated with their generation process will have a uni-directional propagation away from the point of inception. This is why special sound dampening devices can be created that redirect the noise backwards towards the initial source of noise, via specially designed spiral channels, which will reduce the level of the noise perceived by the users.

In general, the pressure waves or radial waves or pseudo-planar waves or focused or non-focused waves or ultrasound, etc. are less powerful than the shockwaves due to the fact that the shockwaves have more energy carried in their higher compressive pressures produced in their compressive phase and larger negative pressure from their tensile phase, negative pressure that can produce more powerful cavitation bubbles in a fluid. On their turn, the pressure waves or radial waves or pseudo-planar waves or focused or non-focused waves or ultrasound, etc. are having a pressure signal flatter, more sinusoidal in shape, and due to their lower positive pressures and smaller values for negative pressures that influences the size of cavitation bubbles, they will put less energy inside the targeted zone.

The pressure waves or radial waves or pseudo-planar waves or focused or non-focused waves or ultrasound, etc. can use reflectors or not, based on a specific application. However, any of them have a preferential direction of propagation, which carries not only their energy, but also the associated noise generated during their generation process. This is why the need of using specially designed devices that can attenuate or dampen the noise associated with pressure waves or radial waves or pseudo-planar waves or focused or non-focused waves or ultrasound, etc.

Overall, the input energy that is used to create shockwaves or pressure waves or radial waves or pseudo-planar waves or focused or non-focused waves or ultrasound, etc. is delivered for the embodiments presented in these inventions of this patent from a power supply in the form of high voltage setting for electrohydraulic and piezoelectric devices and electrical current setting for electromagnetic devices and ultrasonic devices. The explosive devices are activated by currents generated by a power supply and the hydraulic devices use either currents or voltages for their power supply, depending on the way the overpressure is generated. The input energy necessary to produce shockwaves or pressure waves or ultrasound, etc. can be variable with minimal and maximum values. The higher the input energy, the more powerful are the shockwaves or pressure waves or ultrasound, etc. and also the noise associated with their inception.

The power supply functionality and the parameters of the shockwave or pressure wave or ultrasound devices are controlled by a control console/unit, designed to have processors and microprocessors, displays, input/output elements, timers, memory units, remote control devices, independent power unit, etc. Each of these components may include hardware, software, or a combination of hardware and software configured to perform one or more functions associated with providing good functioning of the process that employs the use of the shockwaves or pressure waves or radial waves or pseudo-planar waves or focused or non-focused waves or ultrasound.

It is an objective of the present inventions to disclose different embodiments that can redirect the noise backwards towards the source of it and in this way reducing the overall noise level produced by any type of acoustic waves (shockwaves, pressure waves, radial waves, pseudo-planar waves, focused or non-focused waves, ultrasound, etc.) at their generation and usage, noise that propagates in the surrounding environment. For that, special sound or noise dampening devices can be created to collect a significant portion of the initial noise and redirect it backwards towards the initial source of noise, via specially designed spiral channels, which will reduce the level of the noise perceived by the users.

### BRIEF DESCRIPTION OF THE DRAWINGS

FIG. 1 illustrates the principle of operation for sound dampening applicable to acoustic waves (shockwaves, pressure waves, radial waves, pseudo-planar waves, focused or non-focused waves, ultrasound, etc.), according to one embodiment of the present inventions.

FIG. 2 illustrates an acoustic wave applicator (or wand or probe or emitter, etc.) components, applicator that is connected to its power supply and control console/unit.

FIG. 3 illustrates an acoustic wave applicator including a noise dampening. device or element or component or part using the principle described in FIG. 1, according to one embodiment of the present inventions.

FIG. 4 is a perspective three-dimensional view of short and tall designs for noise dampening devices capable of accommodating different types of acoustic wave applicators, according to embodiments of the present inventions.

FIG. 5 is a cross-sectional view of the short noise dampening device, according to one embodiment of the present inventions.

FIG. 6A is a perspective three-dimensional and sectional view of the short noise dampening device presented in FIG. 5, according to one embodiment of the present inventions.

FIG. 6B is a view from the bottom of the short noise dampening device presented in FIGS. 5 and 6A, according to one embodiment of the present inventions.

FIG. 6C is a perspective three-dimensional and sectional view, seen from the top and with the noise collection upper chamber removed, of the short noise dampening device presented in FIGS. 5, 6A, and 6B, according to one embodiment of the present invention.

FIG. 6D is a cross-sectional view, along the section plane B-B from FIG. 5, of the short noise dampening device presented in FIGS. 5, 6A, 6B, and 6C, according to one embodiment of the present invention.

FIG. 6E is a cross-sectional view, along the section plane A-A from FIG. 5, of the short noise dampening device presented in FIGS. 5, 6A, 6B, 6C, and 6D, according to one embodiment of the present invention.

FIG. 7 is a cross-sectional view of the tall noise dampening device, according to one embodiment of the present invention.

FIG. 8 is a perspective three-dimensional view of the noise collection spiral channels from the noise dampening section that has its outer skin removed for both short and tall noise dampening devices from FIGS. 4, 5, 6A-6E, and 7, according to embodiments of the present invention.

FIG. 9 is a perspective three-dimensional view of an acoustic wave applicator with a transparent representation of the noise dampening device, according to embodiments of the present invention.

FIG. 10 is a perspective three-dimensional view of an acoustic wave applicator with the noise dampening device presented in FIG. 9 that has only the noise dampening section with its outer skin removed, according to embodiments of the present invention.

FIG. 11 is a perspective three-dimensional lateral view of an acoustic wave applicator with the noise dampening device presented in FIGS. 9 and 10 that has only the noise dampening section with its outer skin removed, according to embodiments of the present invention.

### DETAILED DESCRIPTION OF THE INVENTION

Embodiments of the inventions will be described with reference to the accompanying figures, wherein like numbers represent like elements throughout. Further, it is to be understood that the phraseology and terminology used herein is for the purpose of description and should not be regarded as limiting. The use of “including”, “comprising”, or “having” and variations thereof herein is meant to encompass the items listed thereafter and equivalents thereof as well as additional items. The terms “connected”, and “coupled” are used broadly and encompass both direct and indirect mounting, connecting and coupling. Further, “connected” and “coupled” are not restricted to physical or mechanical connections or couplings.

The inventions summarized below and defined by the enumerated claims are better understood by referring to the following detailed description, which is preferably read in conjunction with the accompanying drawing/figure. The detailed description of a particular embodiment is set out to enable one to practice the inventions, it is not intended to limit the enumerated claims, but to serve as a particular example thereof.

The inventions described herein are not intended to be limited to specific embodiments that are provided by way of example but extend to the full scope of such claims of a corresponding issued patent. Also, the list of embodiments presented in this patent is not an exhaustive one and for those skilled in the art, new embodiments, applications, and optimization methods can be realized within the scope of the inventions.

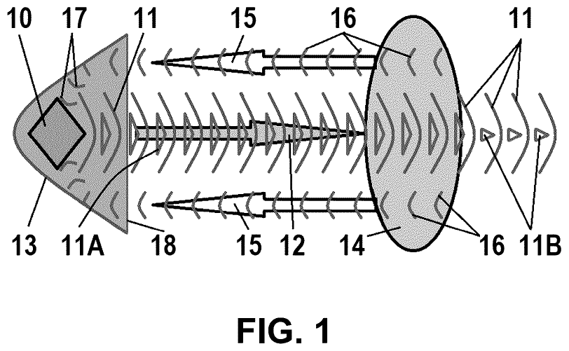

The principle of functioning for the sound dampening device is presented schematically in FIG. 1. It will be appreciated that although descriptions herein may refer to examples including shockwaves and shockwave sources, that other acoustic wave sources and acoustic waves may be similarly implemented. Accordingly, focused shockwaves are produced by a shockwave source 10 positioned inside the reflector 13. The role of the reflector 13 is to direct the shockwaves 11 in a preferred direction towards the targeted zone (not shown in FIG. 1 for simplicity of the figure). During generation of shockwaves, the shockwave source 10 also produce the initial associated noise 11A that propagates, similar to the shockwaves, with the speed of sound and away from the shockwave source 10. Thus, the direction of propagation of the noise produced during the generation of the shockwaves 11 by the shockwave source 10 is the same with the direction of propagation for the shockwave. The shockwaves and noise propagation direction 12 is given by the construction of the reflector 13, and the position or orientation of the reflector aperture 18. In FIG. 1 the shockwaves and noise propagation direction 12 goes from left to right. The noise dampening element 14 is symmetrically positioned on the shockwaves and noise propagation direction 12 and it is designed in such way to let the shockwaves 11 pass through without attenuation. In the same time, the noise dampening element 14 captures the majority of initial associated noise 11A and changes the noise propagation backwards in the form of the backwards redirected noise 16, as indicated by the direction of redirected noise 15. The remaining portion of the initial associated noise 11A is still transmitted forward in the form of reduced transmitted noise 11B. However, the reduced transmitted noise 11B is only a small portion of the initial associated noise 11A, which means that the noise that is perceived by the user is significantly reduced. The backwards redirected noise 16 travel towards the reflector 13 and then it is reflected in the form of reflector redirected noise 17 towards the shockwave source 10. Due to rather opposite direction of the redirected noise 16 and reflector redirected noise 17 relatively to the shockwaves and noise propagation direction 12, a partial cancellation of the subsequent noise generated by the shockwave source 10 is produced, which finally causes a further reduction of the overall noise level. It is important to note that the noise dampening element 14 is doing the reduction in noise level without any attenuation of the energy of the shockwaves, which continue to move undisturbed towards their targeted zone (not shown in FIG. 1 for simplicity). Correspondingly, the same noise reduction done by the noise dampening element 14, it happens when the shockwaves are replaced with pressure waves or radial waves or pseudo-planar waves or focused or non-focused waves or ultrasound, etc.

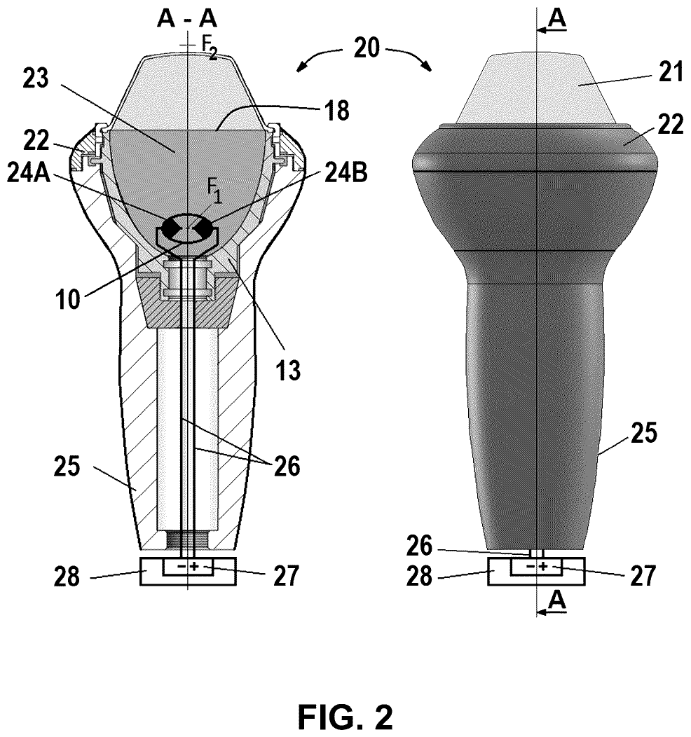

It is an objective of the present inventions to disclose different embodiments of the sound dampening device. Although the embodiments presented in this patent inventions can be applied for any type of acoustic waves (shockwaves, pressure waves, radial waves, pseudo-planar waves, focused or non-focused waves, ultrasound, etc.), one preferable embodiment of a noise dampening device construction and method of use is for a hand-held electrohydraulic shockwave applicator that produces shockwaves, as presented in FIG. 2. Each acoustic pressure focused shockwave generating device or shockwave/acoustic wave applicator 20 has an acoustic shockwave reflector 13 and an applicator/coupling membrane 21. The membrane ring 22 is assembled on the applicator body/handle 25 and its role is to keep the liquid applicator/coupling membrane 21 pressed and fixed against the rim of the reflector 13, which creates the fluid-filled reflector cavity 23. The energy source for the acoustic pressure shockwaves comes from a high voltage power supply 27 that is controlled by a control console/unit 28. The high voltage produced by the high voltage power supply 27 is sent via high voltage cable 26 towards the shockwave source 10 and is applied across a first electrode 24A and second electrode 24B that are immersed in the fluid-filled reflector cavity 23, whereby the membrane 21 seals the fluid and the shockwave source 10 therein. A high voltage applied between the first electrode 24A and second electrode 24B results in an electrical current flowing between the two electrodes. The electrical current increases at an extremely fast rate, in the tens of nanoseconds, while at the same time superheating the fluid in between the first electrode 24A and second electrode 24B to create a plasma bubble. The formation of a plasma bubble in between the first electrode 24A and second electrode 24B occurs at a rate in the tens of nanoseconds, similar to the increasing rate of change of the electrical current. The rise in electrical current creates the front or positive pressure component of the shockwaves. Once the potential voltage between the first electrode 24A and second electrode 24B is no longer supplied or sufficient to support the flow of electrical current and in the moment when the pressure of the fluid-filled reflector cavity 23 surrounding the plasma bubble will be higher than the plasma bubble's internal pressure, that will be the transition process causing the plasma bubble to rapidly collapse creating the negative (cavitation) pressure component of the shockwaves and also the initial associated noise 11A that propagates out of the reflector 13 through the reflector aperture 18. The magnitude of voltage applied between the first electrode 24A and second electrode 24B can be controlled locally by the power supply 27 that is controlled by a control console/unit 28.

The acoustic shockwave reflector 13 shown in FIG. 2 is a partial ellipsoidal reflector. An ellipse is characterized by two focal points, with the property that whatever energy is generated in the first focal point (F₁) can be reflected completely on its surface towards the second focal point (F₂), which is a unique property of this geometry. The acoustic pressure shockwaves 11 (see FIG. 1) produced at the first focal point F₁, as diagramed in FIG. 2, are reflected and focused by the reflector 13 towards the second focal point F₂ of the partial ellipsoid reflector 13. It is the combination of the geometrical design properties of the reflector 13, together with the applied energy created in first focal point F₁, and the placement of the shockwave/acoustic wave applicator 20 relatively to the target of the shockwaves, where usually the second focal point F₂ should be placed. Due to the fact that different pressures fronts (direct or reflected) reach the second focal point F₂ with certain small-timing differences, the acoustic pressure shockwaves 11 are in reality concentrated or focused on a three-dimensional space around second focal point F₂, which is called focal volume (it is not shown in FIG. 2 for simplicity of the figure). Inside the focal volume are found the highest-pressure values for each acoustic pressure shockwave 11, which means that is preferable to position the targeted area in such way to intersects the focal volume that usually is centered around the second focal point F₂. In general, such shockwave/acoustic wave applicators 20 from FIG. 2 are modular and do not need high maintenance.

However, besides the ellipsoidal geometry of the reflector 13 other types of reflector's geometries can be used as parabolic, spheric, cylindrical, conical, pyramidal, tubular, flat, etc., or combination of any of those with two or more geometries present on a specific reflector.

For the shockwave/acoustic wave applicators 20 from FIG. 2, the accumulative energy is the energy level or energy flux density found in F₂ that is dependent of the input energy used by the shockwave source 10 from F₁, the total number of the acoustic pressure shockwaves or pressure wave pulses generated, repetition frequency of the acoustic pressure shockwaves or pressure waves, and special construction of the reflector 13. The input energy, total number of shockwaves (or different acoustic waves in other cases), and their frequency is influencing the total amount of noise that is produced by the generation of the shockwaves 11 (or different acoustic waves in other cases) inside the reflector 13. Thus, the higher the input energy, total number of shockwaves (or different acoustic waves in other cases), and their frequency, the higher it will be the total amount of noise produced during the inception of the shockwaves (or different acoustic waves in other cases) from the shockwave source 10 (or different acoustic waves sources in other cases). The geometry of the reflector 13 can only influence the propagation and focusing of noise in the surrounding volume and not its intensity.

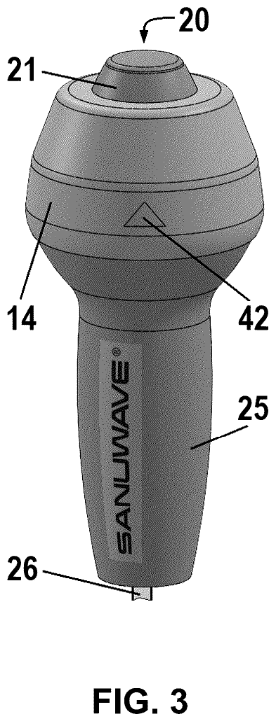

In FIG. 3 is presented an embodiment of an annular noise dampening element 14 with upper opening that sits on top of the shockwave/acoustic wave applicators 20, similar to the ones presented before in FIG. 2. Note that the noise dampening element 14, with at least a top portion of the applicator membrane 21 passing through the upper opening of the dampening element 14, seamlessly hugs the shockwave/acoustic wave applicator 20, and preferably fits on the largest diametral dimension of the applicator body/handle 25, and has direct contact with the applicator/coupling membrane 21 through which the noise dissipates inside the surrounding space.

In various embodiments, a noise dampening element 14 may be made from a variety of materials and creation methods depending on the type of acoustic wave applicator and purpose of the applicator to deliver acoustic waves to a target. For example, for medical uses, any flexible plastic or rubber type materials, including composites, that are safe for human or animal contact, can be used in some embodiments for constructing a noise dampening device. In other medical use embodiments, biological or biological-mimicking materials (such as in case of severe allergy) that are safe for human or animal contact can be used for device 14 construction. In other medical embodiments, organic materials that are safe for human or animal contact can be used to form a noise dampening device 14. In non-medical use embodiments, noise dampening devices 14 may be formed from materials such as flexible or rigid metals including composites, liquid metals, flexible or rigid plastics including composite materials, flexible or rigid rubber type materials including composite materials, flexible or rigid concrete materials including composite materials, stone-including composite materials, flexible or rigid wood materials including composite materials, brick-including composites, carbon composites, sand, ceramics or glass-like materials.

In various embodiments, different creation methods for an acoustic wave noise dampening device 14 include: machining, 3D printing, molding, growing biomedical mimicking materials, forming techniques, pressing, casting and the like.

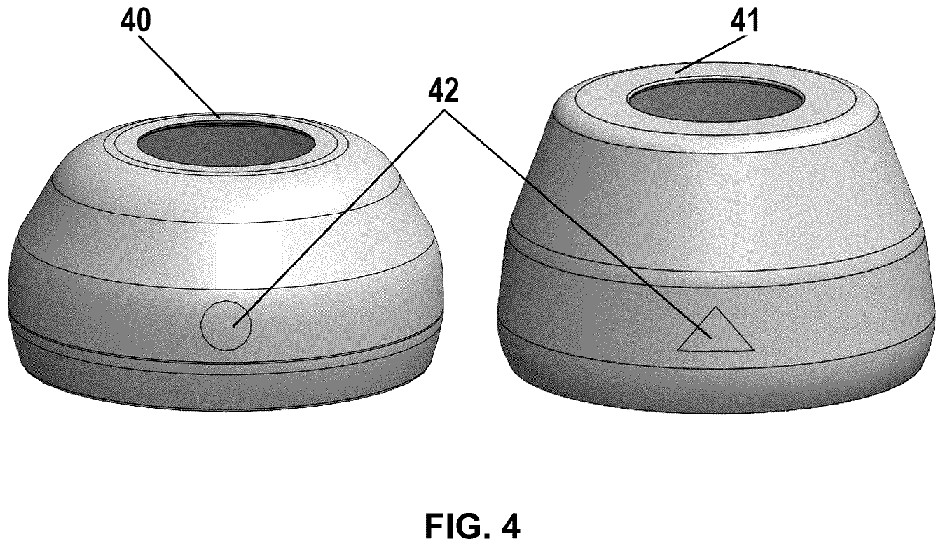

The direct contact of the noise dampening element 14 with the applicator/coupling membrane 21 assures the collection of the majority of the initial associated noise 11A generated at the inception of the shockwaves 11 (see FIG. 1) by the shockwave source 10 from F₁ (see FIG. 2). When the noise dampening element 14 is used, the only noise that goes out through the applicator/coupling membrane 21 is emanating in the form of reduced transmitted noise 11B through the small portion of the applicator/coupling membrane 21 that exists at the top portion of the noise dampening element 14. The shockwave/acoustic wave applicators 20 and their associated applicator/coupling membranes 21 have different geometries due to their respective usage and based on that it will dictate the need for the noise dampening element 14 to also have different constructions and geometries, as can be seen from some embodiments shown in FIGS. 4-8. This is why, there is a need in certain embodiments to have a visual identification symbol 42 that is easy to be seen and understood by the user, as can be seen from FIG. 3. The visual identification symbols 42 can be any geometrical figures, or numbers, or letters, or special symbols.

In various embodiments, noise dampening element 14 couples to, i.e. fits on, the applicator body/handle 25 through various coupling mechanisms, including, for example (and combinations thereof):
Frictional attachment;
Independent multiple screws (such as at least 4);
Dedicated pins in different holes (such as least at least 4);
Dampening element with a thread portion for attachment;
Conical slots;
Bayoneted system;
Magnets;
Velcro (hook and loop material);
Double-sided sticky tape;
Snap fit on tabs;
Electric locking;
Electromagnetic locking;
Clamps (mechanical or motorized);
Dedicated netting;
Temporary adhesive;
In case of multiple applicators there can be multiple dampening elements that are part of a fixture in which the applicators can be slide in and out for usage;
Vacuum system;
Hydraulic locks;
Pneumatic locks;
Computerized locks;
Hinged attachment;
Adhesive tapes; or
Combinations of the foregoing.

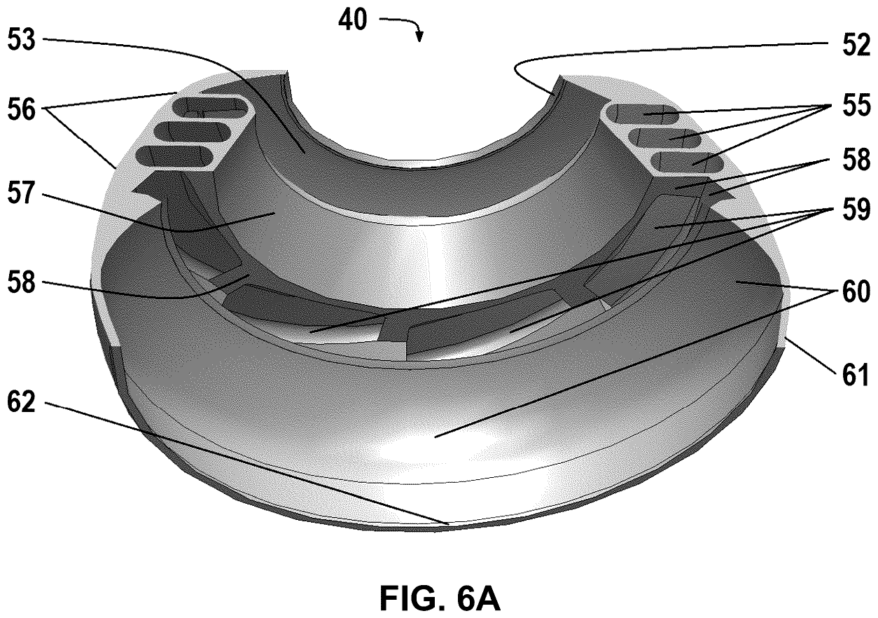

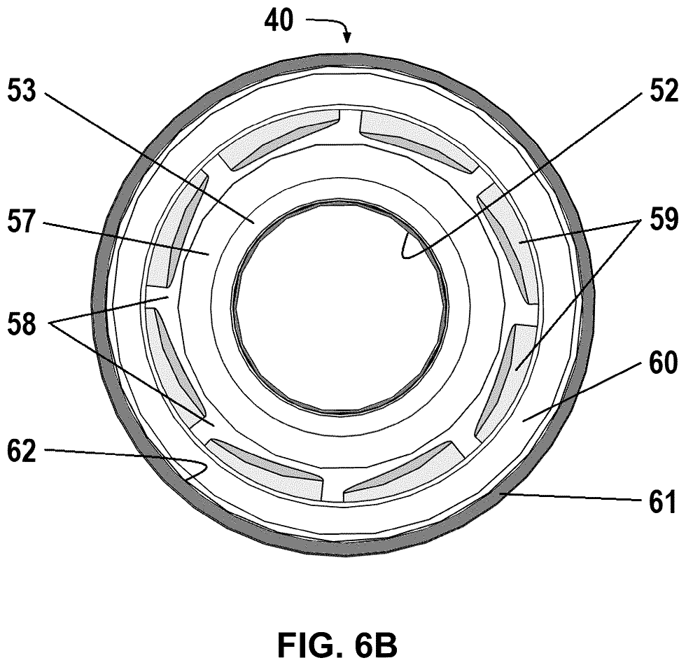

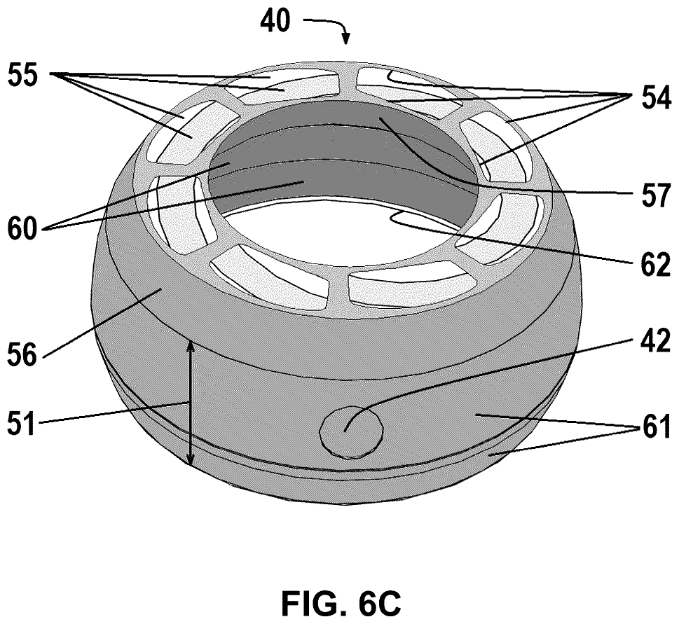

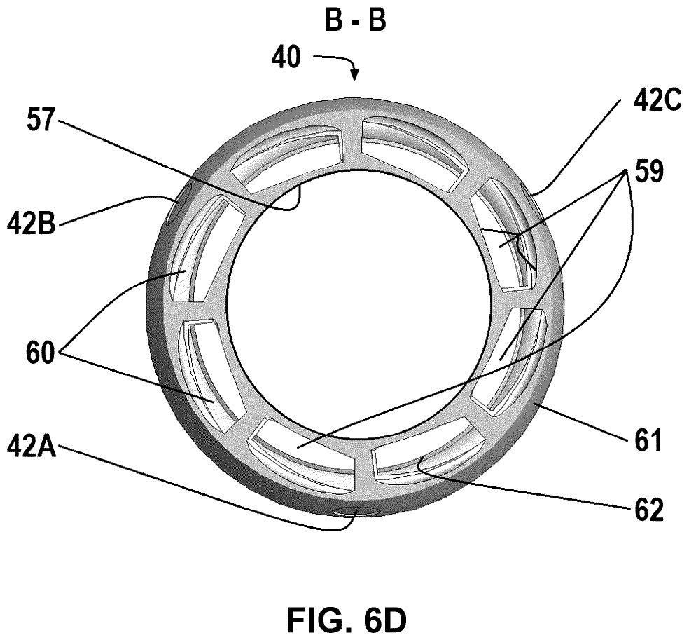

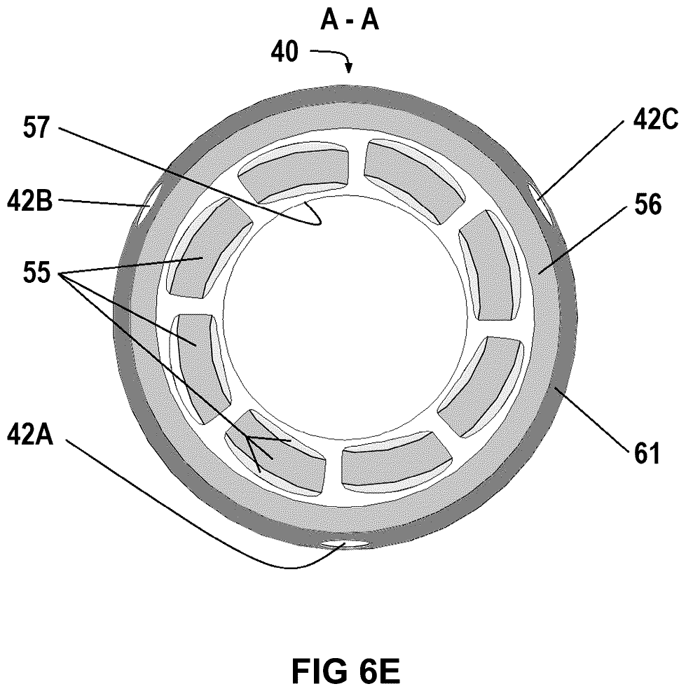

In FIG. 4 are presented two different designs and corresponding embodiments, in general terms similar to the noise dampening element 14 presented in FIG. 3, which are capable to accommodate different types of shockwave/acoustic wave applicators 20. The change in noise dampening element 14 design is dictated by the height of the applicator/coupling membranes 21 and also by the diametral dimension of the reflector aperture 18 (see FIG. 1) that creates the largest dimension of the applicator body/handle 25 where the noise dampening element 14 is attached on the shockwave/acoustic wave applicators 20, as presented in FIG. 3. Out of these two elements, the most influence is given by the height of the applicator/coupling membranes 21 and thus we have a short noise dampening device 40 and a tall noise dampening device 41, as presented in FIG. 4. The difference in between short noise dampening device 40 and a tall noise dampening device 41 is not only in height but also in their geometry, as can be clearly seen from FIG. 4. The users can have multiple designs for the sound dampening devices in their possession and to easily distinguish them on the outside skin of these devices are distinguishable visual identification symbols 42. Furthermore, to avoid a preferential direction where the users can look for these identification symbols 42, in reality for each noise dampening element 14 or more particularly for the short noise dampening device 40 and the tall noise dampening device 41, there are three such symbols separated circumferentially by 120 degrees. As exemplifications, these three symbols are identified as the first visual identification symbol 42A, the second visual identification symbol 42B and the third visual identification symbol 42C that can be seen in FIGS. 6D and 6E.

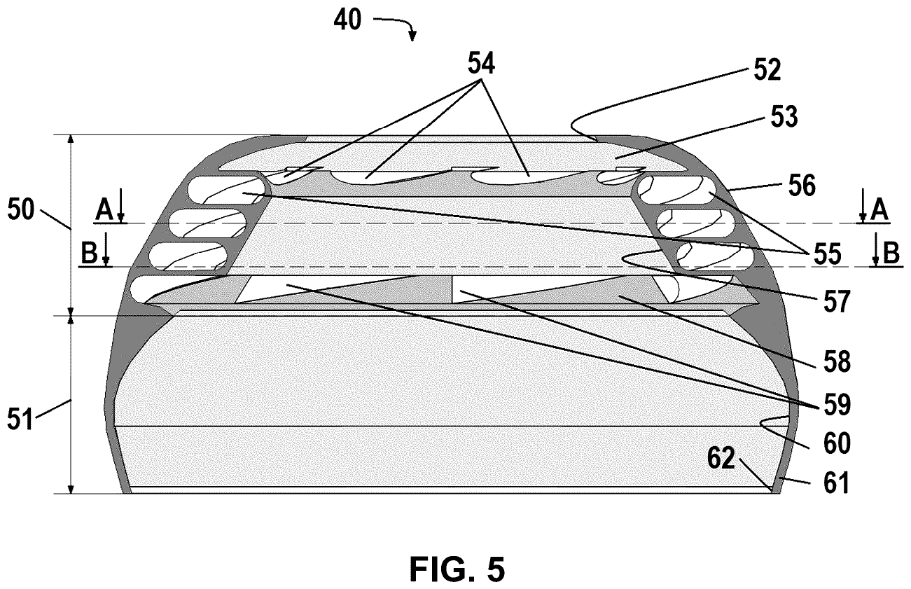

To understand the internal construction of the short noise dampening device 40, in FIG. 5 is presented a cross-sectional representation of such device. Based on the principle of operation presented in FIG. 1, the redirection of the initial associated noise 11A towards its point of origin (shockwave source 10 from FIG. 1) is done for the short noise dampening device 40 using a special noise dampening section 50 and the attachment to the shockwave/acoustic wave applicator 20 in one embodiment is via an applicator attachment section 51 of the short noise dampening device 40. In various embodiments that attachment section 51 facilitates attachment of the short noise dampening device 40 by numerous types of possible attachment mechanisms, including:
Frictional attachment;
Independent multiple screws (such as at least 4);
Dedicated pins in different holes (such as least at least 4);
Dampening element with a thread portion for attachment;
Conical slots;
Bayoneted system;
Magnets;
Velcro (hook and loop material);
Double-sided sticky tape;
Snap fit on tabs;
Electric locking;
Electromagnetic locking;
Clamps (mechanical or motorized);
Dedicated netting;
Temporary adhesive;

In case of multiple applicators there can be multiple dampening elements that are part of a fixture in which the applicators can be slide in and out for usage;
Vacuum system;
Hydraulic locks;
Pneumatic locks;
Computerized locks;
Hinged attachment;
Adhesive tapes; or
Combinations of the foregoing.

The noise dampening section 50 has a noise dampening device upper opening 52, which allows the top portion of the applicator/coupling membranes 21 to pass through and to be with a small portion of it outside the short noise dampening device 40. The noise collection upper chamber 53 is the internal structure of the short noise dampening device 40 where the majority of the forward propagating noise (initial associated noise 11A from FIG. 1) produced by the acoustic waves (shockwaves, pressure waves, radial waves, pseudo-planar waves, focused or non-focused waves, ultrasound, etc.) is collected/reflected and redirected backwards via the noise collection upper openings 54 and associated noise collection spiral channels 55. The number of noise collection spiral channels 55 can be 4 or more, depending on specific requirement of each application where the noise dampening element 14 or more particularly the short noise dampening device 40 and the tall noise dampening device 41 are used. The noise collection spiral channels 55 are placed inside a conical three-dimensional structure that has a spiral channels external conical surface 56 and a spiral channels internal conical surface 57. The spiral channels 55 thread or wrap around each other tightly inside the conical structure and form a complex three-dimensional geometry. Furthermore, the cross section of the noise collection spiral channels 55 for the noise dampening element 14 or more particularly the short noise dampening device 40 and the tall noise dampening device 41 preferentially can be rectangular with round corners, square with round corners, diamond with round corners, triangular with round corners, round, or oval or combination of those. In principle, any geometry or combination of multiple geometries can be used for the cross section of the noise collection spiral channels 55, with the only condition to provide enough volume for the good functioning of the noise collection spiral channels 55 in the constrains of the spiral channels external conical surface 56 and spiral channels internal conical surface 57.

In FIG. 5 it can also be seen that at the bottom of the noise dampening section 50, the noise collection spiral channels 55 end up in noise collection spiral channels lower openings 59 that are placed inside the noise collection lower chamber 58. In the noise collection lower chamber 58, the collected noise is redirected through the applicator/coupling membranes 21 inside the fluid-filled reflector cavity 23 (see FIG. 2) and reflected via reflector 13 towards the point of origin for the noise, which coincide with the shockwave source 10 presented also in FIG. 2. The noise collection upper chamber 53, the noise collection spiral channels 55, and noise collection lower chamber 58 can be filled with any fluid, either liquid or gases or air, with the preference to be filled with air to provide simplicity for the user and avoid extra steps during usage. Such steps might be the filling and emptying of the noise collection upper chamber 53, the noise collection spiral channels 55, and noise collection lower chamber 58 with a fluid or special gases. The applicator attachment section 51 is where the noise dampening element 14 or more particularly the short noise dampening device 40 and the tall noise dampening device 41 are attached and fixed on the shockwave/acoustic wave applicator 20 via the applicator hugging internal surface 60. The applicator attachment section 51 ends at its bottom in the noise dampening device lower opening 62 through which the shockwave/acoustic wave applicator 20 is introduced for accomplishing the coupling with the noise dampening element 14 or more particularly with the short noise dampening device 40 and with the tall noise dampening device 41. The noise dampening device outer skin 61 gives the outer shape of the noise dampening element 14 or more particularly of the short noise dampening device 40 and of the tall noise dampening device 41. All the particularities described previously show that different embodiments presented in these inventions are diverse not only in height and geometry, but also in the internal structure of different sound dampening devices.

In FIGS. 6A-6E the same short noise dampening device 40 is shown in different three-dimensional or two-dimensional views or sectional and cross-sectional representations, to show from different perspectives and in detail the elements mentioned in FIG. 5. Thus, FIG. 6A there is a perspective three-dimensional and sectional representation of the short sound dampening device 40 presented in FIG. 5. In this figure, one can clearly identify and view the noise dampening device upper opening 52, the noise collection upper chamber 53, the noise collection spiral channels 55, the spiral channels external conical surface 56, the spiral channels internal conical surface 57, the noise collection lower chamber 58, the noise collection spiral channels lower openings 59, the applicator hugging internal surface 60, the noise dampening device outer skin 61, and the noise dampening device lower opening 62. In particular, FIG. 6A shows how the noise collection spiral channels lower openings 59 open in the noise collection lower chamber 58.

FIG. 6B is a view from the bottom or from the noise dampening device lower opening 62 perspective of the short sound dampening device 40 presented also in FIGS. 5 and 6A. In this figure, one can clearly identify and view the noise dampening device upper opening 52, the noise collection upper chamber 53, the spiral channels internal conical surface 57, the noise collection lower chamber 58, the noise collection spiral channels lower openings 59, the applicator hugging internal surface 60, the noise dampening device outer skin 61, and the noise dampening device lower opening 62. In FIG. 6B it is interesting to see the trapezoidal-like shape of the noise collection spiral channels lower openings 59, which is also confirmed in FIG. 6D.

FIG. 6C is a perspective three-dimensional and sectional representation, seen from the top and with the noise collection upper chamber removed, of the short sound dampening device 40 presented also in FIGS. 5, 6A, and 6B. In this figure, one can clearly identify and view the noise collection upper openings 54, the noise collection spiral channels 55, the spiral channels external conical surface 56, the spiral channels internal conical surface 57, the applicator hugging internal surface 60, the noise dampening device outer skin 61, and the noise dampening device lower opening 62. In particular, FIG. 6A show also the visual identification symbol 42 (which is a circle in this case) and the trapezoidal-like shape of the noise collection upper openings 54 that collect noise from the noise collection upper chamber 53, as seen in FIGS. 5, 6A, and 6B.

FIG. 6D is a cross-sectional representation, along the section plane B-B from FIG. 5, of the short sound dampening device 40 presented in FIGS. 5, 6A, 6B, and 6C. In this figure, one can clearly identify and view the spiral channels internal conical surface 57, the noise collection spiral channels lower openings 59, the applicator hugging internal surface 60, the noise dampening device outer skin 61, and the noise dampening device lower opening 62. In FIG. 6D it is interesting to see the trapezoidal-like shape of the noise collection spiral channels lower openings 59 and the positioning of the first visual identification symbol 42A, the second visual identification symbol 42B, and the third visual identification symbol 42C (all in the shape of a circle in this case) at 120 degrees circumferential separation.

FIG. 6E is a cross-sectional representation, along the section plane A-A from FIG. 5, of the short sound dampening device 40 presented in FIGS. 5, 6A, 6B, 6C, and 6D. In this figure, one can clearly identify and view the noise collection spiral channels 55, the spiral channels external conical surface 56, the spiral channels internal conical surface 57, and the noise dampening device outer skin 61. In FIG. 6E it is interesting to see the shape of the noise collection spiral channels 55 that is trapezoidal-like and slightly rotated relatively to the spiral channels internal conical surface 57. Also, the positioning of the first visual identification symbol 42A, the second visual identification symbol 42B, and the third visual identification symbol 42C (all in the shape of a circle in this case) at 120 degrees circumferential separation.

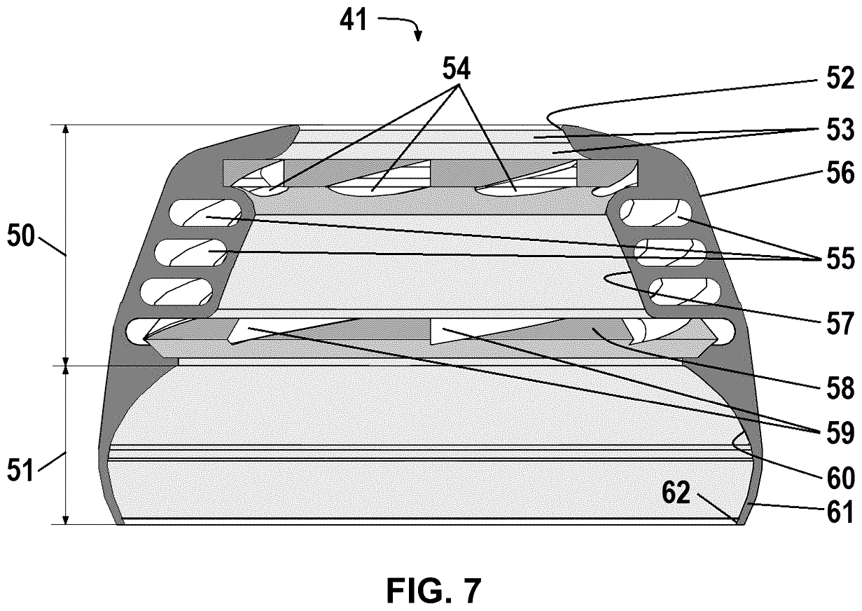

In FIG. 7 is presented a cross-sectional representation of the tall noise dampening device 41. Similar to the explanation provided in FIG. 5 for the short noise dampening device 40 and based on the principle of operation presented in FIG. 1, the redirection of the noise towards its point of origin (shockwave source 10 from FIG. 1) is done for the tall noise dampening device 41 using a special noise dampening section 50 and the attachment to the shockwave/acoustic wave applicator 20 is done via an applicator attachment section 51 of the tall noise dampening device 41. As previously seen In FIG. 5 for the short noise dampening device 40, the tall noise dampening device 41 presented in FIG. 7 has similar elements identified as noise dampening device upper opening 52, noise collection upper chamber 53, noise collection upper openings 54, noise collection spiral channels 55, spiral channels external conical surface 56, spiral channels internal conical surface 57, noise collection lower chamber 58, noise collection spiral channels lower openings 59, applicator hugging internal surface 60, noise dampening device outer skin 61, and the noise dampening device lower opening 62. The functions and uses of all these elements for the tall noise dampening device 41 are the same as described in FIG. 5 for the short noise dampening device 40. However, when compared to the short noise dampening device 40, the tall noise dampening device 41 has the dimension of the noise dampening section 50 much longer and with different geometry of the corresponding portion of the noise dampening device outer skin 61. On its turn, the applicator attachment section 51 for the tall noise dampening device 41 is the same dimension and geometry as for the short noise dampening device 40, if both the tall noise dampening device 41 and the short noise dampening device 40 are used for the same type of shockwave/acoustic wave applicator 20. If different shockwave/acoustic wave applicators 20 (different geometries for the applicator/coupling membrane 21 and membrane ring 22) are accommodating these noise dampening devices, then there will also be a different dimension and geometry for the applicator attachment section 51 for the tall noise dampening device 41 when compared to the short noise dampening device 40. Furthermore, when comparing the tall noise dampening device 41 to the short noise dampening device 40, there are differences in the diametral dimension for the noise dampening device upper opening 52, geometry/shape and dimensions for the noise collection upper chamber 53, noise collection upper openings 54, noise collection spiral channels 55, spiral channels external conical surface 56, spiral channels internal conical surface 57, noise collection lower chamber 58, noise collection spiral channels lower openings 59, and the noise dampening device outer skin 61. The applicator hugging internal surface 60 and the noise dampening device lower opening 62 should be the same or different, if both the tall noise dampening device 41 and the short noise dampening device 40 are used for the same type of shockwave/acoustic wave applicators 20 or not.

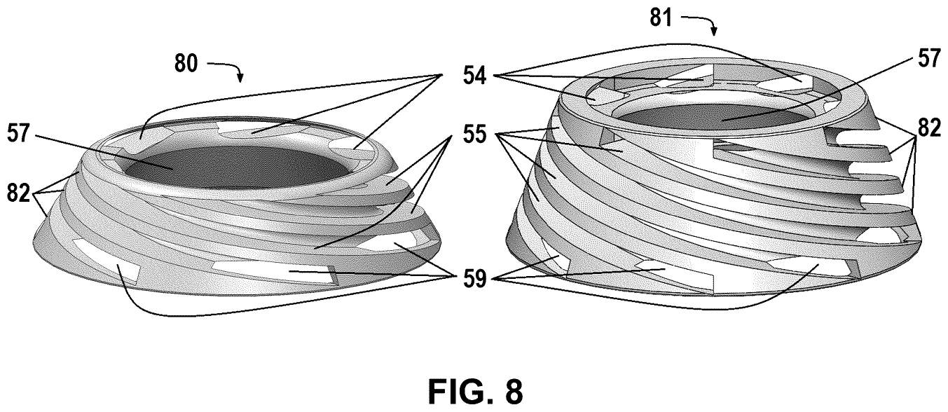

FIG. 8 is a perspective three-dimensional view of the noise collection spiral channels 55 from the noise dampening section 50 (see FIGS. 5 and 7) that has its outer skin removed for both the short sound dampening devices 40 and the tall sound dampening devices 41 presented in FIGS. 4, 5, 6A-6E, and 7. By removing the skin of the noise dampening section 50, skin that is actually the spiral channels external conical surface 56 from FIGS. 5, 6A-6E, and 7, in FIG. 8 are created the short device skinless noise dampening section 80 and the tall device skinless noise dampening section 81. The removal of the skin permits the visualization of the noise collection spiral channels 55 that thread or wrap around each other tightly, which allows the collection of the noise from the upper part of the short device skinless noise dampening section 80 or the tall device skinless noise dampening section 81, using the noise collection upper openings 54, and guide the noise towards the bottom, where the noise collection spiral channels lower openings 59 are located. The noise collection spiral channels 55 wrap inside a conical structure formed in between the spiral channels external conical surface 56 and the spiral channels internal conical surface 57 (see FIGS. 5, 6A-6E, and 7) and in FIG. 8 due to removal of the skin or the spiral channels external conical surface 56, a newly threaded conical surface is formed, which is identified as the spiral channels skinless external conical surface 82.

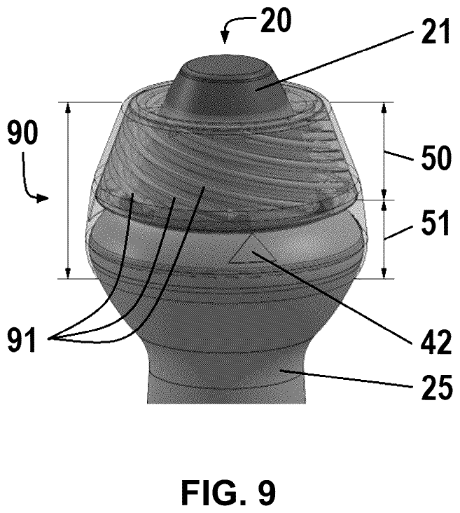

FIG. 9 shows a perspective three-dimensional view of the assembled shockwave/acoustic wave applicator 20 with a transparent noise dampening device 90, to better understand how the noise damping device fits and functions to accomplish noise or sound dampening. The transparent noise dampening device 90 facilitate the viewing on how its two sections, the noise dampening section 50 and the applicator attachment section 51, interact and snuggly fit with the applicator/coupling membrane 21 and the applicator body/handle 25 of the shockwave/acoustic wave applicator 20. The applicator/coupling membrane 21 has the top portion exiting the transparent noise dampening device 90 in such way to not have any interference of the transparent noise dampening device 90 with the surface of the targeted zone (not shown in FIG. 9) that couples with a direct contact with the applicator/coupling membrane 21. The transparent noise dampening device 90 is designed to realize a direct contact with the applicator/coupling membrane 21 at the top portion of the noise dampening section 50 to partially collect the noise that could exit in the environment through the majority of the lateral surface of the applicator/coupling membrane 21. At that point, the partially collected noise is redirected via the noise collection spiral channels 55 as indicated by solid arrows identified as dampening device noise redirections 91, which brings the noise backwards to the bottom of the applicator/coupling membrane 21 where is reinterring the reflector 13 through the reflector aperture 18, as presented in FIG. 1. The applicator attachment section 51 of the transparent noise dampening device 90 tightly hugs the applicator body/handle 25 to secure the transparent noise dampening device 90 on the shockwave/acoustic wave applicator 20 and prevent any unnecessary movements or the detachment of the transparent noise dampening device 90 from the shockwave/acoustic wave applicator 20 during use. Also, note the visual identification symbol 42 for the transparent noise dampening device 90, which in this case is a triangle and it is present on the outer surface/skin of the transparent noise dampening device 90. Even though our comments for FIG. 9 refer to the transparent noise dampening device 90, the functionality, construction and comments are valid for any embodiment/construction of the noise dampening element 14 or more particularly for the short noise dampening device 40 and for the tall noise dampening device 41.

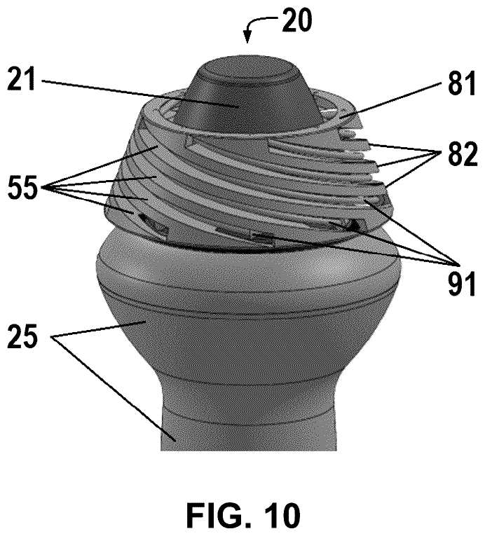

FIG. 10 is a perspective three-dimensional view of a shockwave/acoustic wave applicator 20 in contact with only the noise dampening section 50 of the transparent noise dampening device 90, a was presented in FIG. 9. The noise dampening section 50 has its outer skin removed to easily visualize the noise collection spiral channels 55, the spiral channels skinless external conical surface 82, and the dampening device noise redirections 91 through the noise collection spiral channels 55 towards the bottom of the applicator/coupling membrane 21, the reflector aperture 18 and the reflector 13 (see FIG. 1). Since the tall noise dampening device 41 (see FIG. 4) has its tall device skinless noise dampening section 81 much taller/longer when compared with the corresponding short device skinless noise dampening section 80 (see FIG. 8), to assure a better visualization for the exemplification from FIG. 10, the tall device skinless noise dampening section 81 was chosen to be shown in this figure. The comments presented for FIG. 9 are also valid for FIG. 10. Although our comments for FIG. 10 refer to the transparent noise dampening device 90 as seen it its totality in FIG. 9, the functionality, construction and comments are valid for any embodiment/construction of the noise dampening element 14 or more particularly for the short noise dampening device 40 and for the tall noise dampening device 41.

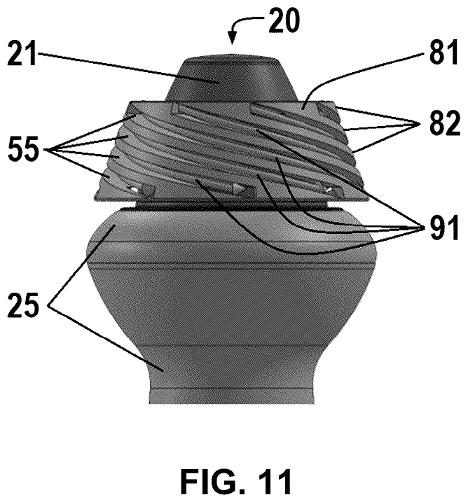

FIG. 11 is a perspective three-dimensional with a lateral view of a shockwave/acoustic wave applicator 20 in contact with the noise dampening section 50 of the transparent noise dampening device 90 presented in FIGS. 9 and 10. As also presented in FIG. 10, the noise dampening section 50 has its outer skin removed to easily visualize the noise collection spiral channels 55, the spiral channels skinless external conical surface 82, and the dampening device noise redirections 91 through the noise collection spiral channels 55 towards the bottom of the applicator/coupling membrane 21, the reflector aperture 18 and the reflector 13 (see FIG. 1). All the other comments presented for FIG. 10 are also valid for FIG. 11.

The following reference Table 1 is provided for the elements described herein:

TABLE 1

| Reference | |
|---|---|
| Number | Element |
| 10 | shockwave source 10 |
| 11 | shockwaves 11 |
| 11A | initial associated noise 11A |
| 11B | reduced transmitted noise 11B |
| 12 | shockwaves and noise propagation direction 12 |
| 13 | reflector 13 |
| 14 | noise dampening element 14 |
| 15 | direction of redirected noise 15 |
| 16 | backwards redirected noise 16 |
| 17 | reflector redirected noise 17 |
| 18 | reflector aperture 18 |
| 20 | shockwave/acoustic wave applicator 20 |
| 21 | applicator/coupling membrane 21 |
| 22 | membrane ring 22 |
| 23 | fluid-filled reflector cavity 23 |
| 24A | first electrode 24A |
| 24B | second electrode 24B |
| 25 | applicator body/handle 25 |
| 26 | high voltage cable 26 |
| 27 | power supply 27 |
| 28 | control console/unit 28 |
| 40 | short noise dampening device 40 |
| 41 | tall noise dampening device 41 |
| 42 | visual identification symbols 42 |
| 42A | first visual identification symbol 42A |
| 42B | second visual identification symbol 42B |
| 42C | third visual identification symbol 42C |
| 50 | noise dampening section 50 |
| 51 | applicator attachment section 51 |
| 52 | noise dampening device upper opening 52 |
| 53 | noise collection upper chamber 53 |
| 54 | noise collection upper openings 54 |
| 55 | noise collection spiral channels 55 |
| 56 | spiral channels external conical surface 56 |
| 57 | spiral channels internal conical surface 57 |
| 58 | noise collection lower chamber 58 |
| 59 | noise collection spiral channels lower openings 59 |
| 60 | applicator hugging internal surface 60 |
| 61 | noise dampening device outer skin 61 |
| 62 | noise dampening device lower opening 62 |
| 80 | short device skinless noise dampening section 80 |
| 81 | tall device skinless noise dampening section 81 |
| 82 | spiral channels skinless external conical surface 82 |
| 90 | transparent noise dampening device 90 |
| 91 | dampening device noise redirection 91 |

Various embodiments of the invention have been described. It will, however, be evident that various modifications and changes may be made thereto, and additional embodiments may be implemented, without departing from the broader scope of the invention as set forth by the claims. This specification is to be regarded in an illustrative rather than a restrictive sense.

What is claimed is:

1. An acoustic wave noise dampening device comprising an annular shape and having an upper opening configured to receive at least a top portion of an acoustic wave applicator membrane and further configured to reduce acoustic wave noise.

2. The acoustic wave noise dampening device of claim 1, further comprising a plurality of additional openings apart from the upper opening configured to receive at least a top portion of an acoustic wave applicator membrane.

3. The acoustic wave noise dampening device of claim 2, wherein the plurality of additional openings includes a plurality of upper openings and a plurality of lower openings.

4. The acoustic wave noise dampening device of claim 3, further comprising a plurality of channels.

5. The acoustic wave noise dampening device of claim 4, further comprising a plurality of spiral channels.

6. The acoustic wave noise dampening device of claim 5, wherein one or more channels of the plurality of spiral channels include an external conical surface and an internal conical surface.

7. The acoustic wave noise dampening device of claim 1, further comprising a plurality of channels.

8. The acoustic wave noise dampening device of claim 7, further comprising a plurality of spiral channels.

9. The acoustic wave noise dampening device of claim 8, wherein one or more channels of the plurality of spiral channels include an external conical surface and an internal conical surface.

10. An apparatus comprising an acoustic wave source and fluid sealed within an applicator membrane and a noise dampening element surrounding at least a portion of the applicator membrane.

11. The apparatus of claim 10, wherein a top portion of the applicator membrane exits from a top portion of the noise dampening element.

12. The apparatus of claim 11, wherein the acoustic wave source and fluid are sealed by the applicator membrane in a reflector cavity.

13. The apparatus of claim 10, wherein the acoustic wave source and fluid are sealed by the applicator membrane in a reflector cavity.

14. The apparatus of claim 13, wherein the noise dampening element includes a plurality of openings.

15. The apparatus of claim 14, wherein the noise dampening element includes a plurality of upper openings and a plurality of lower openings.

16. The apparatus of claim 15, wherein the noise dampening element includes a plurality of channels.

17. The apparatus of claim 16, wherein the plurality of channels are spiral channels.

18. The apparatus of claim 17, wherein one or more channels of the plurality of channels include an external conical surface and an internal conical surface.

19. The apparatus of claim 10, wherein the noise dampening element includes one or more channels.

20. The apparatus of claim 19, wherein at least one of the one or more channels includes a fluid.
